(19) United States
(12) Patent Application Publication
(10) Pub. No.: US 2026/0263159 A1
(43) Pub. Date: Sep. 10, 2026

(54) SYSTEM AND METHOD FOR BONE TRACKING

(71) Applicant: Kyniska Robotics SAS, Saint-Martin-d’Hères (FR)

(72) Inventors: Daniel Elizondo Moreno, Grenoble (FR); Jean-François Larue, Saint-Quentin-sur-Isère (FR); Stéphane Lavallée

(73) Assignee: Kyniska Robotics SAS, Saint-Martin-d’Hères (FR)

(21) Appl. No.: 19/074,179

(22) Filed: Mar. 7, 2025

(51) Int. Cl. A61B 34/20; A61B 34/10

(52) U.S. Cl. CPC A61B 34/20; A61B 34/10; A61B 2034/105; A61B 2034/2057

(57) ABSTRACT

A system for tracking a rigid anatomical structure includes an acquisition device configured to acquire imaging data of a trackable surface, the trackable surface being a portion of the rigid anatomical structure and including one or more characteristic features, a tracking device configured to track a pose of the acquisition device with respect to a reference coordinate system, and one or more processors. The one or more processors are configured to receive imaging data of the trackable surface, receive tracking data and determine a pose of the acquisition device with respect to the reference coordinate system, track a pose of the one or more characteristic features of the trackable surface within the imaging data, based on the pose of the one or more characteristic features, determine a pose of the trackable surface with respect to the acquisition device, and determine a pose of the rigid anatomical structure.

### FIELD

This disclosure relates to systems and methods for tracking a rigid anatomical structure.

### BACKGROUND

Computer-assisted surgery (CAS) aims to increase the precision and positive outcomes of traditional surgical procedures. During CAS, for instance, a robot arm or other device can be used to position, navigate, or actuate a surgical device, such as a drill, in the surgical site of the patient's anatomy. For a CAS procedure to be effective then, a system must know the precise position and orientation of the surgical device and the precise position and orientation of the anatomical structure to be operated on. Otherwise, the surgical device could not be precisely navigated and actuated in relation to the anatomical structure to be operated on. Currently, systems can track in real-time the position and orientation of the anatomical structure to be operated on and the surgical device. The precision of surgical actions during a CAS mainly depends on accurate tracking.

Therefore, improved systems and methods for tracking a position and orientation of an anatomical structure to be operated on are still needed.

### SUMMARY

The present disclosure relates to a system for tracking a rigid anatomical structure in computer-assisted surgery, including: an acquisition device configured to acquire imaging data of a trackable surface, the trackable surface being a portion of the rigid anatomical structure, the trackable surface including one or more characteristic features identifiable in the imaging data; a tracking device configured to track a pose of the acquisition device with respect to a reference coordinate system; and one or more processors communicatively coupled to the acquisition device and the tracking device, configured to: receive imaging data of the trackable surface from the acquisition device; receive tracking data from the tracking device and determine a pose of the acquisition device with respect to the reference coordinate system based on the tracking data; track a pose of the one or more characteristic features of the trackable surface within the imaging data; based on the pose of the one or more characteristic features, determine a pose of the trackable surface with respect to the acquisition device; and determine a pose of the rigid anatomical structure with respect to the reference coordinate system based on the pose of the acquisition device with respect to the reference coordinate system and the pose of the trackable surface with respect to the acquisition device.

The present disclosure relates to a system, including: one or more processors; and one or more storage mediums having encoded thereon executable instructions that, when executed by the one or more processors, cause the one or more processors to: receive imaging data of a trackable surface, wherein the trackable surface: is a portion of a rigid anatomical structure to be operated on; and includes one or more characteristic features; track a pose of the one or more characteristic features in the imaging data; and determine, based on the pose of the one or more characteristic features, a pose of the rigid anatomical structure.

The present disclosure relates to a method for tracking a rigid anatomical structure, the method including: identifying a trackable surface, the trackable surface being a portion of the rigid anatomical structure, the trackable surface including one or more characteristic features; receiving imaging data of the trackable surface, the one or more characteristic features being identifiable in the imaging data; tracking a pose of the one or more characteristic features of the trackable surface within the imaging data; and based on the pose of the one or more characteristic features, determining a pose of the rigid anatomical structure.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 depicts a method of tracking a pose of a rigid anatomical structure, according to one or more embodiments herein;

FIG. 2 depicts a method of selecting a trackable surface, according to one or more embodiments herein;

FIG. 3A depicts a method of developing a trackability map, according to one or more embodiments herein;

FIG. 3B depicts a method of using a trackability map to select a trackable surface, according to one or more embodiments herein;

FIG. 3C depicts a generation of a trackability map, according to one or more embodiments herein;

FIG. 4A and FIG. 4B depict example arrangements of an image acquisition device, using a support, according to one or more embodiments herein;

FIG. 5A and FIG. 5B depict example arrangements of an image acquisition device, using a frame coupled to a surgical table, according to one or more embodiments herein;

FIG. 6A and FIG. 6B depict example arrangements of an image acquisition device, using a robotic arm, according to one or more embodiments herein;

FIG. 7A and FIG. 7B depict example arrangements of an image acquisition device, with an image acquisition device placed in an incision, according to one or more embodiments herein;

FIG. 8A and FIG. 8B depict example arrangements of an image acquisition device, using an endoscopic camera, according to one or more embodiments herein;

FIG. 9 depicts examples of natural characteristic features of the trackable surface, according to one or more embodiments herein;

FIG. 10 depicts examples of artificially emphasized characteristic features of the trackable surface, according to one or more embodiments herein;

FIG. 11A and FIG. 11B depict example methods of tracking a pose of a trackable surface, according to one or more embodiments herein; and

FIG. 12 depicts an exemplary computing device, according to one or more embodiments herein.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure provides systems and methods for tracking a position and orientation (e.g., a pose) of an anatomical structure to be operated on, during CAS for instance. Current systems and methods for tracking a pose of an anatomical structure have their shortcomings. Therefore, improved systems and methods for tracking a pose of an anatomical structure during a surgical operation are required.

The present disclosure provides such improved systems and methods. Particularly, the present disclosure relates to tracking a pose of only a portion of a rigid anatomical structure and using the pose of the portion of the rigid anatomical structure to determine a pose of the entire anatomical structure. In some embodiments, the rigid anatomical structure can be, for instance, bone, cartilage, or another anatomical structure expected not to, or to minimally, deform during a surgery. In some embodiments, the rigid anatomical structure can be a structure to be operated on during a surgical intervention, such as CAS. In some embodiments, the portion of the rigid anatomical structure that is tracked is generally a portion of the surface of the rigid anatomical structure. It should be appreciated that the portion of the surface of the rigid anatomical structure is smaller than the whole of the surface of the anatomical structure and can be considered a sub-region of the rigid anatomical structure. The portion of the surface of the rigid anatomical structure can be described as a trackable surface herein. While the specific embodiment herein is discussed with reference to tracking a pose of the rigid anatomical structure, it should be appreciated that in some embodiments, the systems and methods herein could be used to track at least one of a position or an orientation of the rigid anatomical structure.

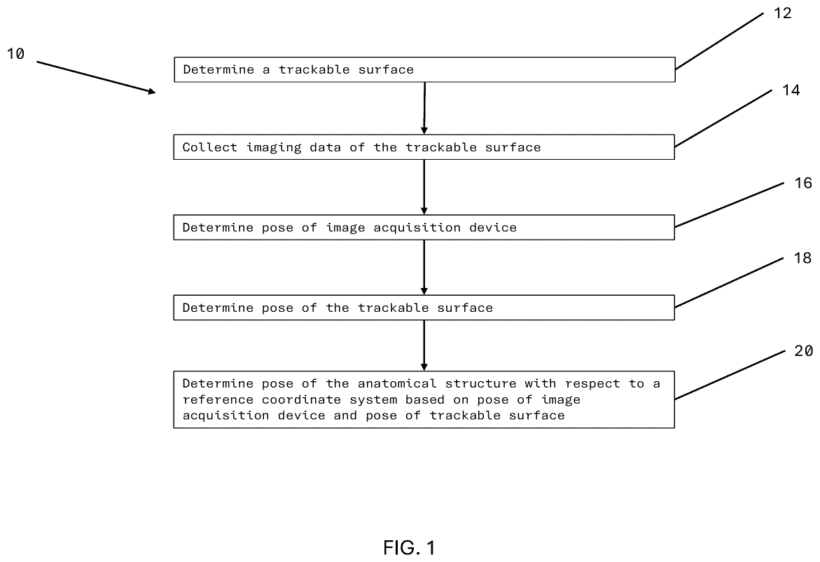

A high-level method 10 of tracking a pose of a rigid anatomical structure is depicted in FIG. 1. In some embodiments, in a step 12, the method can include determining a trackable surface of the rigid anatomical structure. In some embodiments, the trackable surface is only a portion of the entire surface of the anatomical structure. In some embodiments, the trackable surface is roughly a 10 mm×10 mm area of the surface of the rigid anatomical structure. In some embodiments, the trackable surface is roughly a 7.5 mm×7.5 mm area of the surface of the rigid anatomical structure. In some embodiments, the trackable surface is roughly a 5 mm×5 mm area of the surface of the rigid anatomical structure. In some embodiments, the trackable surface is roughly a 2.5 mm×2.5 mm area of the surface of the rigid anatomical structure. In some embodiments, the trackable surface is roughly a 1 mm×1 mm area of the surface of the rigid anatomical structure. The trackable surface can be sized such that the trackable surface contains a sufficient number of characteristic features (discussed in further detail below) to allow for the tracking of the trackable surface. In some embodiments, determining a trackable surface of the rigid anatomical structure can include identifying one or more characteristic features of a portion of the entire surface of the anatomical structure. Particularly, in some embodiments, the one or more characteristic features are identifiable in imaging data acquired of the rigid anatomical structure. In some embodiments, the acquired imaging data can be of only the portion of the entire surface of the anatomical structure that is the trackable surface. In some embodiments, the characteristic features are natural features of the trackable surface of the rigid anatomical structure. For example, the characteristic features can be a porosity of a surface of the trackable surface, a fibrosity or striation of the surface of the trackable surface, or micro-reliefs in the surface of the trackable surface. In some embodiments, the characteristic features can be reinforced, or emphasized, with artificial features added to or implemented on the trackable surface. For example, in some embodiments, the characteristic features can be reinforced from an alteration of a surface condition of the trackable surface by one or more techniques such as drilling, milling, burring, engraving, embossing, cauterizing, applying ink. In some embodiments, the characteristic features can be reinforced, or emphasized using structured light using various patterns. The artificial features can improve the trackability of the trackable surface. In some embodiments, the one or more characteristic features appear as image texture in imaging data acquired of the trackable surface. That is, the one or more characteristic features can be identified and tracked in acquired imaging data of the trackable surface by appearing as image texture information in the acquired imaging data. In embodiments where the image acquisition device used to collect the imaging data is a 2D image acquisition device, the image texture can be a 2D graphical feature. In embodiments, where the image acquisition device used to collect the imaging data is a 3D image acquisition device, the image texture can be a 3D graphical feature. In some embodiments, the image texture may include small-scale structures perceivable in an image based on spatial arrangement of colors or intensities, for instance. The trackability of the trackable surface can be thought of as the ability to identify the characteristic features in imaging data and the ability to determine a pose of the characteristic features from the imaging data. If a portion of the surface of the rigid anatomical structure is deemed to include one or more trackable characteristic features, then the portion of the surface can be determined as, or tagged as, the trackable surface of the anatomical structure. In some embodiments, the trackable surface is determined not just based on the surface condition of the trackable surface (e.g., characteristic features), but also based on operating parameters of the planned surgical intervention of the rigid anatomical structure. The operating parameters can be, for example, planning data for the surgical intervention, the surgical history of the patient, and the environment (such as the layout of the operating room) the surgery is to be performed in.

In some embodiments, in a step 14 of the method 10, imaging data of the trackable surface can be collected. Because the trackable surface is just a portion of the entire surface of the rigid anatomical structure, in some embodiments, imaging data can be collected with a smaller frame of view of just the trackable surface, instead of collecting imaging data on the entire rigid anatomical structure. That is to say, in some embodiments, the field of view of an image acquisition device can be sized such that the entire trackable surface is in the field of view, but that some or all of the remaining surface of the anatomical structure that is outside of the trackable surface is not in the field of view of the image acquisition device. It should be appreciated that, in some embodiments, the entire rigid anatomical structure could be in the field of view of the image acquisition device, but that only imaging data related to the trackable surface would be analyzed, as discussed below. In some embodiments, the imaging data can be acquired with any suitable image acquisition device.

In some embodiments, in a step 16 of the method 10, a pose of the image acquisition device can be determined. In some embodiments, a marker having a detectable and trackable pose can be coupled to the image acquisition device for tracking a pose of the image acquisition device. In some embodiments, a pose of the image acquisition device can be determined via an optical localization system. In such embodiments, one or more optical brackets (e.g., markers) can be attached to the image acquisition device and tracked by an optical tracker. In some embodiments, a pose of the image acquisition device can be determined via an electromagnetic localization system. In such embodiments, one or more electromagnetic trackers (e.g., markers) can be attached to the image acquisition device and tracked by an electromagnetic emitter. In some embodiments, in which the image acquisition device is coupled to a robotic arm, for instance, the pose of the image acquisition device can be determined through inverse kinematics. The pose of the image acquisition device can be determined relative a reference global coordinate system (e.g., a global coordinate system based on the operating room the surgery is performed in).

In some embodiments, a step 18 of the method 10 can include tracking a pose of the trackable surface of the rigid anatomical structure. In some embodiments, the pose of the trackable surface can be determined from imaging data of the trackable surface. Tracking a pose of the trackable surface can first include tracking a pose of the one or more characteristic features of the trackable surface. For instance, a change in pose of the one or more characteristic features can be determined between a first image of the trackable surface (e.g., imaging data at a first point in time) and a second image of the trackable surface (e.g., imaging data at a second point in time). Because the characteristic features can have a known pose relative the rest of the trackable surface, a pose of the trackable surface can be determined from a pose of the one or more characteristic features.

In some embodiments, a step 20 of the method can include determining a pose of the rigid anatomical structure. Because the anatomical structure is rigid and the trackable surface is part of the anatomical structure, and the trackable surface is in a known position relative the rest of the anatomical structure, the pose of the anatomical structure can be determined from the pose of the trackable surface. In some embodiments, the pose of the rigid anatomical structure can be determined with respect to the reference coordinate system. That is, because the pose of the image acquisition device is known with respect to the reference coordinate system and the pose of the trackable surface (including the characteristic features) is known with respect to the image acquisition device, the pose of the trackable surface can in turn be determined relative the reference coordinate system. Further, because the pose of the rigid anatomical structure is known with respect to the trackable surface, the pose of the rigid anatomical structure is in turn known with respect to the reference coordinate system. Once the pose of the rigid anatomical structure is known, the pose of the portion of the rigid anatomical structure to be operated on can be precisely determined, and one or more surgical tools can be precisely moved and operated with respect to the portion of the rigid anatomical structure to be operated on. For instance, one or more surgical robots can navigate surgical tools to precisely operate on the portion of the rigid anatomical structure to be operated on, as it will have precisely known coordinates in the global reference coordinate system.

The increased precision of determining the pose of the rigid anatomical structure can increase the precision of the control of a surgical tool with respect to the rigid anatomical structure during CAS. In some embodiments, because only the trackable surface needs to be tracked, a smaller portion of the rigid anatomical surface needs to be visible to the image acquisition device, making the tracking process less invasive, as large portions (or the entirety of) the bone need not be exposed. Additionally, because only the trackable surface needs to be tracked, as opposed to a larger surface or the entire rigid anatomical structure, the likelihood of a person or equipment interrupting the line-of-sight of the image acquisition device to the trackable surface can be reduced. Because the tracking is based on characteristic features of the rigid anatomical structure, optical brackets or electromagnetic pins need not be driven into the bone, reducing patient trauma. Details with respect to the systems and methods disclosed herein will be discussed with respect to the following figures.

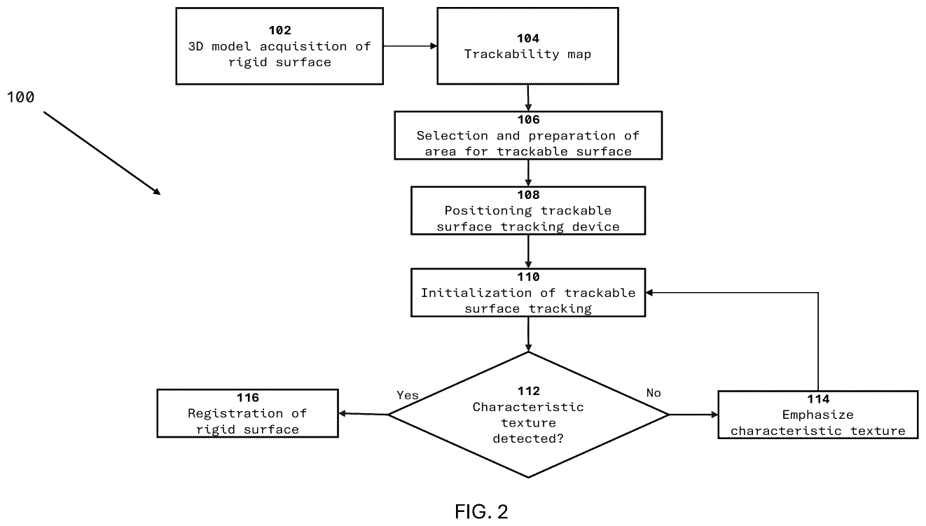

Referring now to FIG. 2, a method 100 is depicted. The method 100 can be used to determine a portion of the rigid anatomical structure to be the tracking surface. In some embodiments, in a step 102, a 3D model of the rigid anatomical structure can be obtained. In some embodiments, the 3D model of the rigid anatomical structure can be acquired from a volumetric imaging system, such as computer tomography (CT) or magnetic resonance imaging (MRI). In some embodiments, the 3D model of the rigid anatomical structure can be used by a physician in planning a surgical procedure on the rigid anatomical structure. In some embodiments, the 3D model of the rigid anatomical structure can reveal an image texture of the surface of the rigid anatomical structure. The image texture of the trackable surface of the rigid anatomical structure can be characteristic features of the trackable surface of the rigid anatomical structure, as described in greater detail below. That is, the characteristic features of the rigid anatomical structure can be identifiable as image textures. In some embodiments, the 3D model of the rigid anatomical structure can be generated or received by one or more processors.

In some embodiments, in a step 104 of the method 100, a trackability map of the rigid anatomical structure can be generated and used. The trackability map can generally be calculated to reveal optimal portions of (e.g., locations on) the surface of the rigid anatomical structure to be used as the trackable surface. In some embodiments, one or more processors can consider a variety of factors to generate the trackability map of the rigid anatomical surface. In some embodiments, the trackability map can be displayed as a graphical indication of the degree of suitability of different portions of the rigid anatomical structure for being the trackable surface (e.g., in terms of colors, gradients, etc.). In some embodiments, the graphical indication can be presented on the graphical image of the rigid anatomical structure, such as the 3D model of the rigid anatomical structure generated in step 102. The trackability map can assign a trackability score to different portions of the surface of the rigid anatomical structure, where the trackability score indicates the degree of trackability of the portion. The trackability score can be assigned in any suitable manner, such as numerically or graphically.

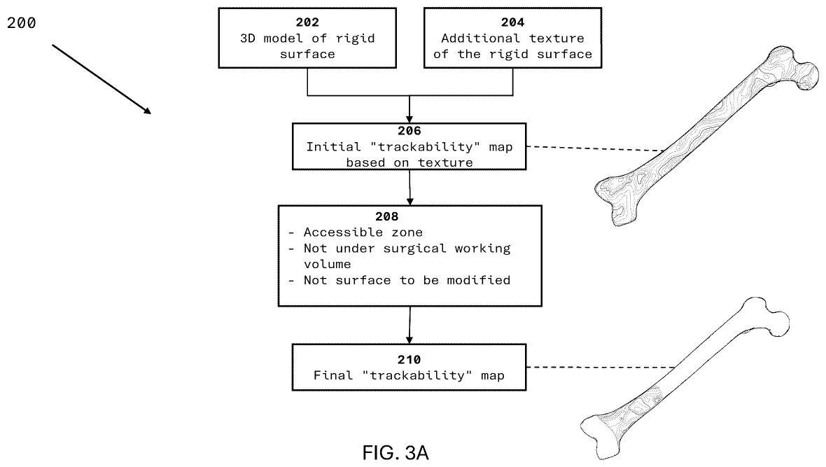

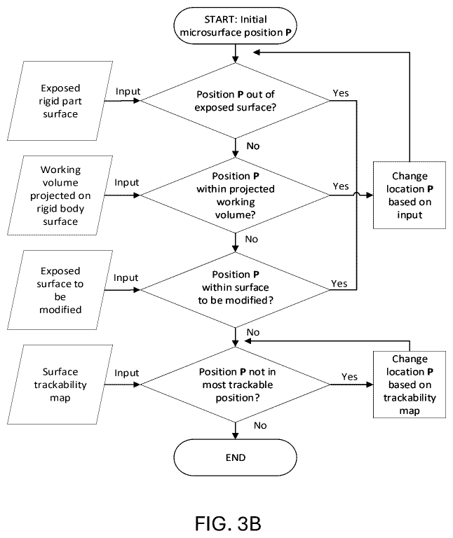

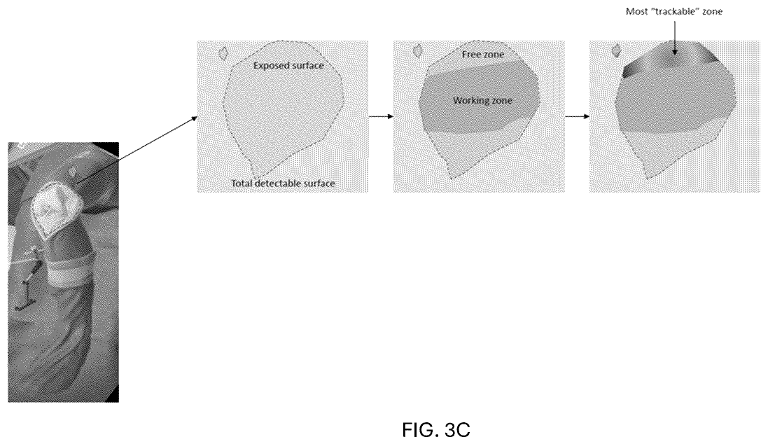

Referring now to FIG. 3A, a method 200 of developing a trackability map is shown in more detail. In some embodiments, in a step 202, one or more processors can receive the 3D model of the rigid anatomical structure generated in step 102 of the method 100, for instance. As noted above, in some embodiments, the 3D model of the rigid anatomical structure can reveal image texture information of the surface of the rigid anatomical structure. In some embodiments, in a step 204, one or more processors can receive additional image texture information of the rigid anatomical structure. The additional image texture information can be derived, for instance, from MRI images of the rigid anatomical structure, CT images of the rigid anatomical structure, cone beam computed tomography scan (CBCT) images of the rigid anatomical structure, or the like. In some embodiments, in a step 206, an initial trackability map of the rigid anatomical structure is formed based on the image textures of the rigid anatomical structure. In some embodiments, the initial trackability map can show initial determinations of the suitability (e.g., the trackability) of different portions of the surface of rigid anatomical structure to be deemed the trackable surface. In some embodiments, the trackable surface should have a particular texture in the images that will allow for easier identification and detection of pose of the trackable surface during the surgical operation, as discussed in greater detail below. Therefore, a portion of the surface that displays a first image texture can have a first trackability score in the initial trackability map, and a portion of the surface that displays a second image texture can have a second trackability score in the initial trackability map.

In some embodiments, in a step 208 of the method 200, the initial trackability map can be modified based on one or more secondary factors affecting trackability. In some embodiments, a trackability score can be assigned to various secondary factors and such score can be used to modify the trackability map. In some embodiments, the trackability score may be weighted based on importance of the one or more secondary factors being scored. In some embodiments, the one or more secondary factors are input by a user, such as a surgeon performing the operation on the rigid anatomical structure. While surgeons are specifically used as an example throughout the disclosure, it should be appreciated that any individual, including any medical care provider, system technician or any other person present in the operating room during the procedure, could be a user of the system. In some embodiments, the one or more secondary factors can be information on the surgical intervention, or operation, to be performed on the rigid anatomical structure. In some embodiments, a detailed surgical plan developed by the surgeon can be analyzed by one or more processors to update the trackability map. For instance, information on the type of surgery to be performed, the particular intervention to be completed on the rigid anatomical structure, the angle of approach the surgeon plans to take to the rigid anatomical structure, the tools to be used during the surgery, etc. can be used to update the trackability map. In some embodiments, the one or more secondary factors can include the environment in which the surgery will be performed. In some embodiments, the one or more secondary factors can include a patient's medical or surgical history. For instance, if a patient has previously had surgery on the same rigid anatomical structure, the rigid anatomical structure may have been altered in the previous surgery, leaving useful characteristic features in an area of the anatomical structure that can be identified and tracked. Generally, the trackability map (e.g., the trackability score of different portions of the surface of the rigid anatomical structure) can be updated based on the accessibility of the portion of the surface of the rigid anatomical structure in the surgical room (for example, so the trackable surface is positioned such as it is in the field of view of an image acquisition device), the working volume for the planned surgical intervention on the rigid anatomical structure (for example, such that the field of view is not blocked during the procedure), and the planned modifications of the rigid anatomical structure during the surgical intervention (for example, such that the characteristic features being tracked are not altered or removed during the procedure).

With respect to accessibility in the surgical room, this factor can consider, for example, the location of the trackable surface such that it can easily be placed in the field of view of an image acquisition device. For example, in some embodiments, the trackability map would not recommend a portion of the surface of the rigid anatomical structure that is not accessible or visible to an acquisition device as the trackable surface. That is, in some embodiments, a portion of the surface of the rigid anatomical structure that is not accessible or visible in the surgical room would receive a low trackability score, and in some embodiments, a trackability score of zero. For instance, if the surgery is to be performed on a femur, and during the surgery, the leg is to be in a supine straight position, the trackability map would assign a low trackability score (e.g., a zero score) to a portion of the back of the femur. In such cases, the back of the femur can be facing and supported by the operating table and is not realistically available to collect imaging data on during the surgery. In cases where the planned type of the surgery is mostly endoscopic (i.e. arthroscopy), the proposed trackable surface can be under the skin area that must be prepared with a skin incision to place the image acquisition device.

With respect to the working volume of the planned surgical intervention, this factor can consider, for example, the probability of the trackable surface being blocked, and, fi so, for what time duration, from the field of view of an image acquisition device during the procedure. For example, in some embodiments, the trackability map would not recommend a portion of the surface of the rigid anatomical structure that is in the volume of space where the surgeon will be working during the operation to be the trackable surface. That is, in some embodiments, a portion of the surface of the rigid anatomical structure that is directly in the volume of space the surgeon would be working would receive a low trackability score, and in some embodiments, a trackability score of zero. This can ensure, for instance, that the line-of-sight between the image acquisition device and the eventually determined trackable surface is not interrupted during the operation. For instance, the working volume can be the volume of space the surgeon's hands and the surgical tools will be placed to perform the surgery. In some embodiments, the trackable surface will be as far as possible from the working volume to avoid the possibility of image obstruction during tracking. In cases where the planned type of surgery is open (e.g., the rigid anatomical surface is exposed), the proposed trackable surface can be out of the working region (e.g., uncovered by the working volume).

With respect to the planned modifications of the rigid anatomical structure, this factor can consider, for example, to what extent the trackable surface (e.g., characteristic features) are remains the same throughout the duration of the procedure. For example, in some embodiments, the trackability map would not recommend a portion of the surface of the rigid anatomical structure that is in an area to be altered or impacted during the planned surgical intervention to be the trackable surface. That is, in some embodiments, a portion of the surface of the rigid anatomical structure that is to be modified during the surgery would receive a low trackability score, and in some embodiments, a trackability score of zero. For instance, the trackable surface should not be in a portion of the rigid anatomical structure to be cut, to receive an implant (e.g., a plate or screw), or to otherwise be modified, either permanently or temporarily. Such modification of a portion of the rigid anatomical structure inherently modifies the characteristic features of the portion of the rigid anatomical structure and the image texture of the portion of the rigid anatomical structure in the imaging data. This can ensure the continued identification and tracking of characteristic features in the trackable surface, as opposed to current markerless tracking methods based on the shape of a bone to be operated on.

In some embodiments, in a step 210, a final trackability map can be determined. In some embodiments, one or more processors can use the above criteria to update the initial trackability map. The updated trackability map can recommend only some portions of the rigid anatomical structure as being suitable trackable surfaces. The portions can have a suitable image texture, be available in the surgical room, be outside the working volume, and not be planned to be modified. The situation can be described as an optimization problem constrained by the previously described criteria and whose cost-function to minimize is the probability to undermine an effective tracking of the trackable surface. Algorithms using machine learning such as fuzzy logic, simplex, or neural networks can solve this task. It is worth noting that, in absence of the additional texture information coming from images, such as in step 204, other tools might help to evaluate the potential areas to place the trackable surface on the rigid anatomical structure, such as a Gauss map or other tools derived from machine learning.

In some embodiments, after the final trackability map is generated in step 210 of the method 200, a surgeon can analyze the trackability map to select the optimal portion of the rigid anatomical structure to be the trackable surface. For instance, the surgeon can identify and select the portion of the surface of the rigid anatomical structure having the highest trackability score to be the trackable surface. In some embodiments, after the final trackability map is generated in step 210 of the method 200, one or more processors can analyze the trackability map to suggest the optimal portion of the rigid anatomical structure to be the trackable surface (e.g., the portion with the highest trackability score), which a surgeon can then select or confirm. In some embodiments, after the final trackability map is generated in step 210 of the method 200, one or more processors can analyze the trackability map to automatically select the optimal portion of the rigid anatomical structure to be the trackable surface (e.g., the portion with the highest trackability score).

For instance, referring to FIG. 3B, a flowchart generally showing the process of selecting a portion of the surface of the rigid anatomical structure as the trackable surface, according to some embodiments, is shown. In some embodiments, if a portion of the surface is not exposable or accessible in the surgical room, then the trackability map would not offer this portion as a potential trackable surface (e.g., the trackability map could assign the portion a trackability score of zero). In some embodiments, if a portion of the surface is within the working volume of the planned surgery, then the trackability map would not offer this portion as a potential trackable surface (e.g., the trackability map could assign the portion a trackability score of zero). In some embodiments, if a portion of the surface is within the area of the rigid anatomical structure to be modified, then the trackability map would not offer this portion as a potential trackable surface (e.g., the trackability map could assign the portion a trackability score of zero). In some embodiments, the remaining portions of the surface of the rigid anatomical structure, being potential sites for the trackable surface, can be ranked or otherwise denoted based on their trackability (e.g., can be given a non-zero trackability score). In some embodiments, the portion of the surface of the rigid anatomical structure selected to be the trackable surface can have the highest trackability score on the trackability map.

Referring to FIG. 3C, an example trackability map is depicted, excluding un-exposed, or inaccessible, portions of the rigid anatomical structure, portions of the rigid anatomical structure that are in the working volume, and portions of the surface of the rigid anatomical structure to be modified during the surgery. The remaining, potential portions of the surface of the rigid anatomical structure to be the trackable surface are designated in terms of their trackability (by color on the map, for example). The most trackable portion of the surface will be based on the type of surgery to be performed and the particular planned approach to be taken during the surgery, the available field of view of the image acquisition device, and any texture information gathered on the surface of the rigid anatomical structure.

Referring again to FIG. 2, in some embodiments, in a step 106 of the method 100, as discussed above, a user or one or more processors can select the trackable surface. In some embodiments, also in step 106, a surgeon or user can prepare the anatomical area for the determined trackable surface. For instance, a surgeon might make an incision or arthroscopic hole in the skin of the patient to expose the determined trackable surface of the rigid anatomical structure. The incision or hole can be made such that an image acquisition device can be suitably placed to gather imaging data on the trackable surface, as described in greater detail below.

In some embodiments, in a step 108 of the method 100, the image acquisition device can be placed for tracking the pose of the trackable surface. The image acquisition device can be any device suitable for collecting imaging data of the trackable surface. Merely as examples, the image acquisition device can be a mini-camera, a stereo-camera, a 3D camera, an RGB-D camera, an endoscopic camera, a LiDAR camera, or an ultrasonic probe. An RGB-D camera can provide both color and depth information and can be particularly suited for embodiments where the characteristic features of the trackable surface have a variation in depth. In some embodiments, the image acquisition device can be a single camera. In some embodiments, the image acquisition device can include at least two cameras. Generally, the image acquisition device provides imaging data that includes image texture information of the trackable surface. In some embodiments, the image texture information is derived from characteristic features of the trackable surface. Examples of image acquisition devices and setups will be described below.

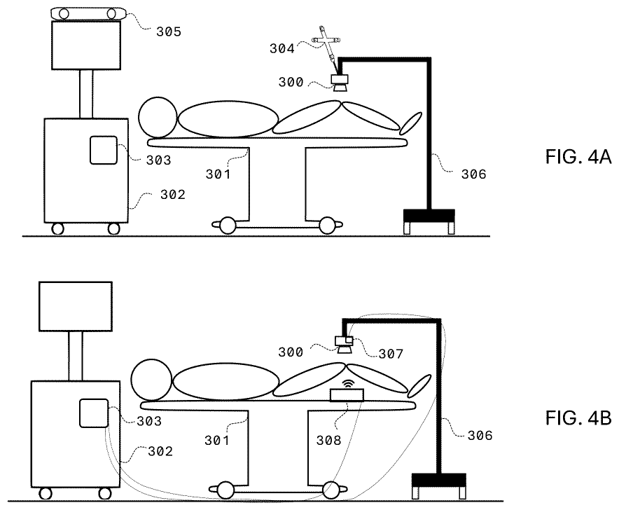

Referring now to FIG. 4A, an example arrangement of an image acquisition device 300 is depicted. In some embodiments, the image acquisition device 300 can be positioned in an operating room having a surgical table 301 and a control unit 302 in which a localization system 303 is attached. In some embodiments, the image acquisition device 300 can be attached to a support 306. In some embodiments, the image acquisition device 300 can have an optical bracket 304 coupled to the image acquisition device 300. In some embodiments, the optical bracket 304 is detectable and trackable by an optical camera 305 communicatively coupled to the localization system 303. In general, the image acquisition device 300 can be positioned such that the trackable surface of the rigid anatomical structure is in the field of view of the image acquisition device 300. In some embodiments, the image acquisition device 300 can be moved during a surgery to maintain the trackable surface in the field of view of the image acquisition device (for instance if the limb containing the rigid anatomical structure is manipulated by the surgeon during the operation). In some embodiments, the localization system can determine a pose of the image acquisition device 300 with reference to a global reference coordinate system based on the pose of the optical bracket. Referring now to FIG. 4B, another example arrangement of an image acquisition device is depicted. The example depicted in FIG. 4B differs from that depicted in FIG. 4A, in that the pose of the image acquisition device 300 is tracked electromagnetically instead of optically. In some embodiments, an electromagnetic tracker 307 is coupled to the image acquisition device 300 and is communicatively coupled to the localization system 303. In some embodiments, an electromagnetic emitter 308 is positioned near the surgical site and communicatively coupled to the localization system 303. The electromagnetic tracker 307 is configured to receive an electromagnetic field, and the emitter 308 is configured to emit the electromagnetic field. The tracking system 303 is configured to determine, from the electromagnetic field received by the electromagnetic tracker 307, the relative pose of the image acquisition device 300 with respect to the global reference coordinate system.

While optical and electromagnetic tracking are discussed in detail herein for the purposes of tracking the image acquisition device, it should be appreciated that any known method for tracking pose can be used to track the pose of the image acquisition device. For instance, visual markers, inertial measuring, or the like can be used to determine a pose of the image acquisition device.

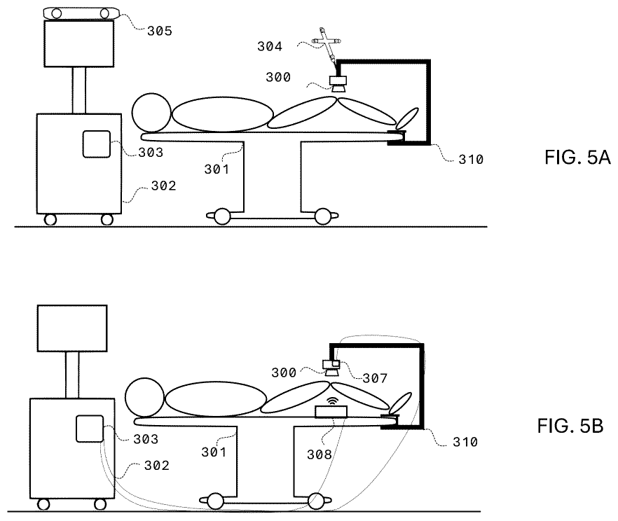

Referring now to FIG. 5A and FIG. 5B, another example arrangement of an image acquisition device 300 is depicted. The arrangements depicted in FIGS. 5A and 5B can largely resemble the arrangements described in FIGS. 4A and 4B, respectively. In the arrangements depicted in FIGS. 5A and 5B, the image acquisition device 300 can be supported by a frame 310 coupled to the surgical table 301.

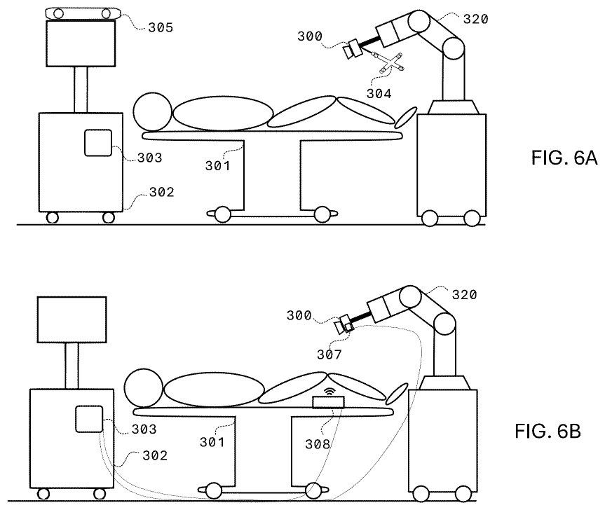

Referring now to FIG. 6A and FIG. 6B, another example arrangement of an image acquisition device 300 is depicted. The arrangements depicted in FIGS. 6A and 6B can largely resemble the arrangements described in FIGS. 4A and 4B, respectively. In the arrangements depicted in FIGS. 5A and 5B, the image acquisition device 300 can be supported by a robot arm 320. In some embodiments, one or more processors can be configured to automatically move the robot arm 320 to maintain the trackable surface in the field of view of the image acquisition device 300. In some embodiments, a user can provide instructions to one or more processors to move the robot arm 320 to maintain the trackable surface in the field of view of the image acquisition device 300. In some embodiments, the robot arm can be instructed by one or more processors to continually move the image acquisition device as the rigid anatomical structure moves. In some embodiments, one or more other imaging devices can be positioned in the surgical room, and based on the imaging data gathered from the one or more other imaging devices, one or more processors can track the hands of a surgeon or one or more tools in the working volume. Based on the tracked body parts or tools in the surgical room, the one or more processors can instruct the robot arm to move the image acquisition device such that its field of view to the trackable surface is not obstructed. FIG. 6A shows an embodiment where the pose of the image acquisition device is tracked optically. FIG. 6B shows an embodiment where the pose of the image acquisition device is tracked electromagnetically. In some embodiments, the pose of the image acquisition device 300 can be determined via inverse kinematics. For instance, in some embodiments, the image acquisition device 300 can be rigidly coupled to the distal end of the robotic arm 320. One or processors can be configured to determine the pose of the distal end of the robotic arm 320 through inverse kinematics. For instance, the one or more processors configured for directing the movement of the robotic arm 320 can know the pose of the distal end of the robotic arm 320 based on control signals provided by the one or more processors, and can therefore determine the pose of the image acquisition device 300 by it being rigidly coupled to the distal end of the robotic arm 320.

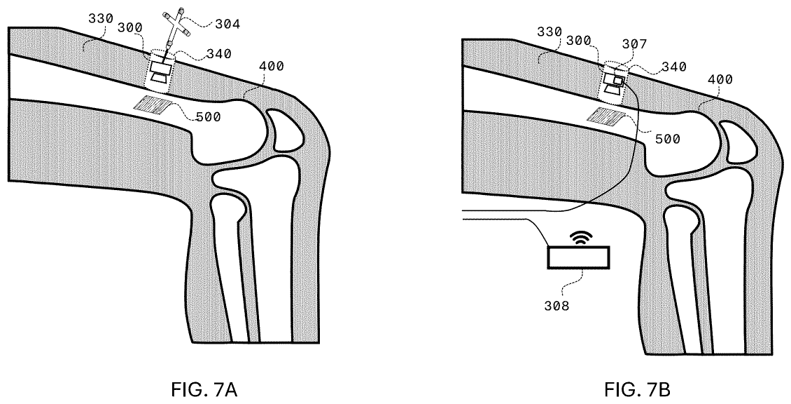

Referring now to FIGS. 7A and 7B, another example arrangement of an image acquisition device 300 is depicted. The arrangements depicted in FIGS. 7A and 7B can largely resemble the arrangements described in FIGS. 4A and 4B, respectively. In the embodiments shown in FIGS. 7A and 7B, a small incision can be made in soft tissue 330 surrounding the rigid anatomical structure 400. In some embodiments, the incision can be positioned generally above the trackable surface 500 to be tracked. In some embodiments, the image acquisition device 300 can be inserted into the incision such that the trackable surface 500 is in the field of view of the of the image acquisition device 300. In some embodiments, the image acquisition device 300, itself, can maintain the incision open. In some embodiments, a tube 340 can be placed inside the incision to maintain the incision open. In some embodiments, the image acquisition device 300 can be placed in the tube 340. In some embodiments, a retractor can be used to maintain the incision open. As mentioned above, the trackable surface 500 can be spaced a distance away from the working volume and the portions of the rigid anatomical structure to be modified during the surgical intervention. In such cases, the incision the image acquisition device 300 is placed through can be different from the main working incision that the surgeon will make to perform the operation on the rigid anatomical structure 400. FIG. 7A shows an embodiment where the pose of the image acquisition device is tracked optically. FIG. 7B shows an embodiment where the pose of the image acquisition device is tracked electromagnetically.

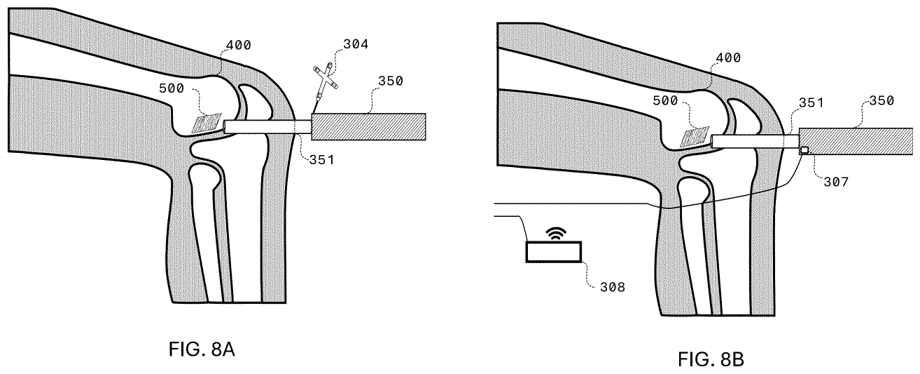

Referring now to FIGS. 8A and 8B, another example arrangement of an image acquisition device 300 is depicted. The arrangements depicted in FIGS. 8A and 8B can largely resemble the arrangements described in FIGS. 4A and 4B, respectively. In the embodiments shown in FIGS. 8A and 8B, the image acquisition device is an endoscopic camera 350. The endoscopic camera 350 can be slid through a small incision 351 to place the trackable surface 500 in the field of view of the endoscopic camera 350. As mentioned above, the trackable surface 500 can be spaced a distance away from the working volume and the portions of the rigid anatomical structure to be modified during the surgical intervention. In such cases, the incision 351 the endoscopic camera 350 is placed through can be different from the main working incision that the surgeon will make to perform the operation on the rigid anatomical structure 400. FIG. 8A shows an embodiment where the pose of the image acquisition device is tracked optically. FIG. 8B shows an embodiment where the pose of the image acquisition device is tracked electromagnetically.

In any of the above examples, the image acquisition device can include a light-emitting device. The light-emitting device can guarantee homogeneous illumination on the region of interest, including the trackable surface. In some embodiments, the homogenous illumination includes spectrum beyond the visible light, such as infrared or UV, for the cases in which the characteristic features to be tracked becomes visible under these wavelengths. In some embodiments, the light-emitting device can emit structured light, which can produce a visual pattern or polarized light on the trackable surface. As described further below, the use of the light-emitting device can enhance the characteristic features for a better acquisition rate in the imaging data of the trackable surface. In particular, the use of the light-emitting device can help to identify and determine the pose of the identifiable features. In some embodiments, the use of the light-emitting device can assist in capturing additional identifiable features, such as the ones that may be difficult to capture without light. Typically, the image acquisition device can be positioned close to the trackable surface, but a distance where the field-of-view of the image acquisition device is enough to capture the full trackable surface as well as its characteristic features. In all of the above examples, the pose of the image acquisition device can be tracked with respect to a global reference coordinate system.

Referring again to FIG. 2, in some embodiments, in a step 110 of the method 100, the system for tracking the trackable surface can be initialized. Specifically, referring to a step 112 of the method 100, one or more processors can collect imaging data, from the image acquisition device, of the selected portion of the surface of the rigid anatomical structure that is the trackable surface. The one or more processors can analyze the imaging data to determine whether the characteristic features of the trackable surface are detectable in the imaging data. In some embodiments, the one or more processors can assign a detectability score to the characteristic features of the trackable surface, indicating the degree the characteristic features are detectable. The characteristic features are physical features of the trackable surface of the rigid anatomical structure and are identified as image texture in the imaging data.

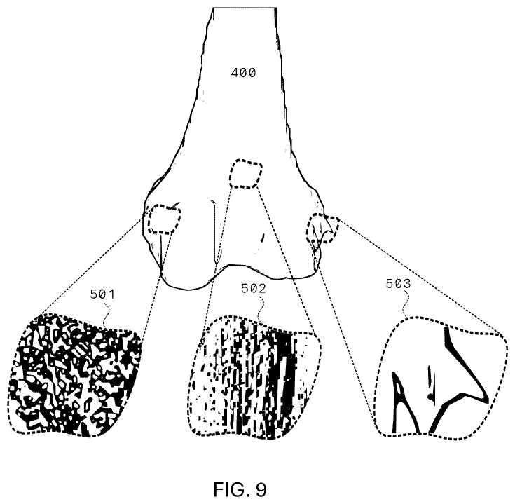

Referring to FIG. 9, examples of natural characteristic features of the trackable surface are shown. For instance, the natural characteristic features of the trackable surface that can be detected as image texture in the imaging data include porosity 501, fibrosity or striation 502, or micro-reliefs 503. In some embodiments, the natural characteristic features can include a grain of the trackable surface. In some embodiments, the natural characteristic features can include imperfections such as osteophytes, which are the result of the grains and mineral depositions that could happen on the osseous cell scaffold. In some embodiments, a natural characteristic feature can relate to a surface feature of a portion of the rigid anatomical structure that was naturally found on the surface of the rigid anatomical structure (e.g., was not added to the surface of the rigid anatomical structure by a user). In some embodiments, the characteristic features of the trackable surface can be pre-existing artificial characteristic features. In some embodiments, pre-existing artificial characteristic features can relate to artificial features that are not added or reinforced for the purposes of tracking the pose of the trackable surface during a current operation, but were instead added to the rigid anatomical structure during a previous operation. Merely as an example, a plate or screw implanted in the rigid anatomical structure to fix a prior fracture of the anatomical structure can be a pre-existing artificial characteristic feature of the trackable surface.

In some embodiments, where the imaging data is two-dimensional, the characteristic features of the trackable surface can appear as image texture in the imaging data, and more specifically as a two-dimensional graphical pattern identifiable in the imaging data. In some embodiments, the two-dimensional graphical pattern can be an intensity, a contrast, a gradient, or a color. In some embodiments, where the imaging data is three-dimensional, the characteristic features of the trackable surface can appear as image texture in the imaging data, and more specifically as a three-dimensional feature identifiable in the imaging data. In some embodiments, the three-dimensional feature can be a depth.

Referring again to FIG. 2, in some embodiments, in a step 116 of the method 100, if the characteristic features of the trackable surface are sufficiently identifiable in the imaging data (e.g., have a sufficiently high detectability score), the method 100 can proceed to registration of the rigid anatomical structure. In some embodiments, if the characteristic features of the trackable surface are not sufficiently identifiable in the imaging data (e.g., have a detectability score beneath a threshold), then at step 114 of the method 100, the characteristic features can be reinforced or emphasized by a surgeon.

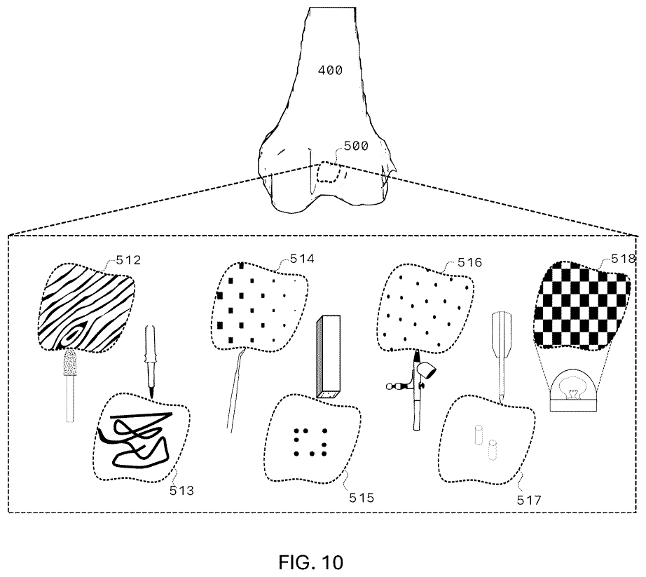

Referring to FIG. 10, examples of artificially emphasized characteristic features are depicted. In some embodiments, the artificially emphasized characteristic feature can result from an alteration of a surface condition of the trackable surface by at least one of drilling, milling, burring, engraving, embossing, cauterizing, or applying ink. In some embodiments, the artificially emphasized characteristic feature can result from an alteration of a surface condition of the trackable surface by the insertion of pins. In some embodiments, the pins can superficially applied to the trackable surface. In some embodiments, an artificially emphasized characteristic feature is emphasized with a mechanical alteration using a power tool such as drilling, milling or burring 512. In some embodiments, an artificially emphasized characteristic feature is emphasized with the application of regular or irregular bio-compatible ink or graphite patterns 513. In some embodiments, an artificially emphasized characteristic feature is emphasized with cauterization using a diathermy tip 514. In some embodiments, an artificially emphasized characteristic feature is emphasized with stamping, engraving or embossing a predefined texture pattern 515. In some embodiments, an artificially emphasized characteristic is artificially emphasized with applying a random pattern of ink droplets 516. In some embodiments, the ink used can be one used on surgical markers, tattoo ink, or any other ink compatible with the trackable surface. In some embodiments, the ink can be of any distinguishable color within the extended visible light spectrum (from infrared to UV). An example could be glowing ink under UV light. In some embodiments, the application of the ink can be by tracing regular know patterns, such as visual marks, tracing irregular patterns using graphite or other type of ink, or spraying ink droplets to create random patterns. Ink can enhance the natural 3D characteristic features on the trackable surface, as the ink will not be applied evenly on the surface depending on bumps or creases, for example, on the surface of the rigid anatomical layer. In some embodiments, an artificially emphasized characteristic feature is emphasized with inserting small pins, rods, micro-pyramids or any other geometric feature 517. In some embodiments, the geometric feature can be disposed according to a template or known pattern. In some embodiments, the geometric pattern can be distinguishable within the extended visible light spectrum (e.g., from infrared to UV). In some embodiments, the geometric features can be superficially added to the rigid anatomical structure. In some embodiments, an artificially emphasized characteristic feature is emphasized with or using structured or polarized light 518. In some embodiments, the characteristic feature can be artificially emphasized by inserting, sticking, or gluing a small pin, already having a printed visual mark on the trackable surface.

In some embodiments, the action of reinforcing, or emphasizing, the characteristic features can be effectuated either by hand or using automatic tools. For example, for the latter, the creation of an artificially reinforced characteristic feature can be done by a robotic, computer-controlled device that will run the power tool over the trackable surface to produce the reinforced characteristic feature. Another example of an automatic device that could be used is a computer-controlled printer with laser, ink-injection or matrix-dot technology, that can leave an inked or engraved mark over the trackable surface. As another example, in some embodiments, an automatic device can insert the corresponding pins, rods, or geometric features at a programmed location.

Referring back to FIG. 2, following the emphasis of the characteristic features, the method 100 can return to steps 110 and 112 to determine if the emphasized characteristic features are identifiable in the imaging data (e.g., have a sufficiently high detectability score). As above, one or more processors can collect imaging data, from the image acquisition device, of the selected portion of the surface of the rigid anatomical structure that is the trackable surface. The one or more processors can analyze the data to determine whether the artificially emphasized characteristic features of the trackable surface are sufficiently identifiable in the imaging data. In some embodiments, the artificially reinforced characteristic features are, also, physical features of the trackable surface of the rigid anatomical structure and are identified as texture in the imaging data. In some embodiments, where the imaging data is two-dimensional, the artificially reinforced characteristic features of the trackable surface can appear as image texture in the imaging data, and more specifically as a two-dimensional graphical pattern identifiable in the imaging data. In some embodiments, the two-dimensional graphical pattern can be an intensity, a contrast, a gradient, or a color. In some embodiments, where the imaging data is three-dimensional, the artificially reinforced characteristic features of the trackable surface can appear as image texture in the imaging data, and more specifically as a three-dimensional feature identifiable in the imaging data. In some embodiments, the three-dimensional feature can be a depth.

In some embodiments, in the step 116 of the method 100, if the artificially reinforced characteristic features of the trackable surface are sufficiently identifiable in the imaging data (e.g., have a detectability score above a threshold), the method 100 can proceed to registration of the rigid anatomical structure. In some embodiments, if the artificially reinforced characteristic features of the trackable surface are not sufficiently identifiable in the imaging data (e.g., have a detectability score below a threshold), then at step 114 of the method 100, the characteristic features can be further reinforced or emphasized by a surgeon. For instance, the surgeon can apply two or more of the artificial reinforcement methods discussed with respect to FIG. 10 to the trackable surface. The method of artificially reinforcing the characteristic features can continue until the characteristic features are sufficiently identifiable in the imaging data. In some embodiments, it can be desired to minimize the amount of artificial reinforcement of the trackable surface of the rigid anatomical structure, and therefore, the reinforcing process can be iterative until the characteristic features are sufficiently identifiable.

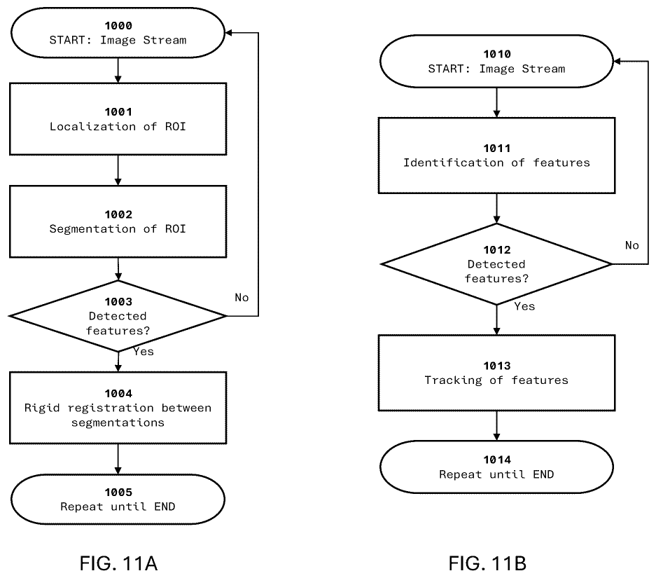

Once it is confirmed that the characteristic features of the trackable surface are sufficiently identifiable in the imaging data, the pose of the characteristic features and the trackable surface (and therefore the rigid anatomical structure) can be tracked in the imaging data. Non-limiting examples of tracking a pose of the characteristic features are discussed with respect to FIG. 11A and FIG. 11B. Generally, tracking a pose of the trackable surface can include identifying like characteristic features in first and second slices of imaging data (e.g., imaging data at a first time and imaging data at a second time) and determining a change in pose of the characteristic features between the first and second slices of imaging data. In some embodiments, a transformation matrix between the pose of the characteristic features in the first slice of imaging data and the pose of the characteristic features in the second slice of imaging data can be determined.

Referring to FIG. 11A, a geometric approach of tracking a pose of the characteristic features is depicted. The geometric approach generally includes representing the trackable surface as a mesh or as a point could in three dimensions. In some embodiments, in a step 1000, imaging data is collected from the image acquisition device. In some embodiments, the imaging data is of only a portion of the rigid anatomical structure (e.g., substantially only of the trackable surface). In some embodiments, in a step 1001, a localization of a region-of-interest (ROI) is automatically effectuated in order to maximize the area to be analyzed with potential characteristic features. That is, the ROI is determined to maximize the number of characteristic features in the ROI. In some embodiments, the ROI is a portion of the trackable surface. In some embodiments, the ROI includes all or substantially all of the trackable surface. In some embodiments, one or more processors can localize the ROI by means of a convolutional neural network (CNN). It should be appreciated that any number of suitable processing methods can be used to localize the ROI. Merely as examples, one or more processors can localize the ROI by means of image processing methods, such as clustering (e.g., using a K-means algorithm), graph methods (e.g., random Markov chains), or segmentation-based object categorization, among others. In some embodiments, in a step 1002, the ROI is segmented, converting the 2D and 3D image texture patterns (e.g., the representations of the characteristic features) in the imaging data of the ROI into micro-shapes that can be tracked. Segmenting the ROI can be considered transforming the ROI from image space to geometrical space. In some embodiments, the ROI can be segmented using a CNN. It should be appreciated that any number of suitable processing methods can be used to segment the ROI. Merely as examples, one or more processors can segment the ROI by means of image processing methods, such as clustering (e.g., using a K-means algorithm), graph methods (e.g., random Markov chains), or segmentation-based object categorization, among others. In some embodiments, segmenting the ROI can produce a mesh or point cloud representation of the ROI. In some embodiments, in a step 1003, evaluation of the number of characteristic features in the segmented ROI is effectuated. For instance, in some embodiments, a number of characteristic features (e.g., micro-shapes that can be tracked) are identified in the ROI. If the number of detected features is above a threshold value, the algorithm can continue to step 1004. If the number of detected features is below a threshold value, the method can return to collecting imaging data at step 1000. In some embodiments, in a step 1004, a rigid registration is performed between a segmented ROI based on imaging data at a second point in time (e.g., a current time) and a segmented ROI based on imaging data at a first point in time (e.g., a previous time). That is, a comparison is made between a micro-shape or characteristic feature in a first segmented ROI and a second segmented ROI. Based on the rigid registration, a transformation matrix is obtained that indicates the change in pose of the characteristic features, and therefore of the trackable surface. In some embodiments, an iterative closest point algorithm can be used for registration purposes. This method is repeated until a command end is received at a step 1005. In some embodiments, the method is continually performed for each slice of imaging data received to track the pose of the trackable surface in real time.

Referring to FIG. 11B, an image approach of tracking a pose of the characteristic features is depicted. As opposed to the geometric approach, the image approach functions only in the image space. One example algorithm that can be used in the image approach of tracking is digital image correlation. The image approach is generally treated as an image matching problem, in which a subset of random features (e.g., a subset of characteristic features) are identified in each image in a stream of images, and the features are compared between a current image and a previous image to determine a motion of the subset of random features. Then, shape functions attached to each of the features of the identified subsets will subsequently describe the change in pose. In some embodiments, in a step 1010, imaging data is collected from the image acquisition device. In some embodiments, the imaging data is of only a portion of the rigid anatomical structure (e.g., substantially only of the trackable surface). In some embodiments, in a step 1012, characteristic features of the trackable surface are identified. Several techniques for the identification of the characteristic features can be used, such as an image-matching subset in which random patterns are identified, a color histogram gradient distribution, or an edge-detection. In some embodiments, a probability distribution of each of the detected characteristic features from a previous and a present image is established. In some embodiments, in a step 1012 of the method, the number of identified characteristic features is evaluated to determine if there are enough identified characteristic features to guarantee the tracking 1012. If the number of detected features is below a threshold value, the method can return to collecting imaging data at step 1010. In some embodiments, in which the number of identified characteristic features is above a threshold, the characteristic features can be tracked at a step 1013. Tracking the pose of the characteristic features generally includes comparing information on an identified characteristic feature in a slice of imaging data at a second point in time (e.g., a current time) and a slice of imaging data at a first point in time (e.g., a previous time). In some embodiments, the tracking consists of tracing the change of the shape of the detected characteristic features. In some cases, brightness is a function of the shape under a constant illumination context. Therefore, a function of brightness can perform as a shape function. Based on the tracking of the characteristic features, a transformation matrix can be calculated that tracks the pose of the trackable surface. This method is repeated until a command end is received at a step 1014. In some embodiments, the method is continually performed for each slice of imaging data received to track the pose of the trackable surface in real time.

As noted above, because the anatomical structure is rigid, the trackable surface is part of the anatomical structure, and the trackable surface is in a known position relative the rest of the anatomical structure, the pose of the anatomical structure can be determined from the pose of the trackable surface. Because the pose of the image acquisition device is known with respect to the reference coordinate system and the pose of the trackable surface (including the characteristic features) is known with respect to the image acquisition device, the pose of the trackable surface can in turn be determined relative the reference coordinate system. Further, because the pose of the rigid anatomical structure is known with respect to the trackable surface, the pose of the rigid anatomical structure can be determined with respect to the reference coordinate system.

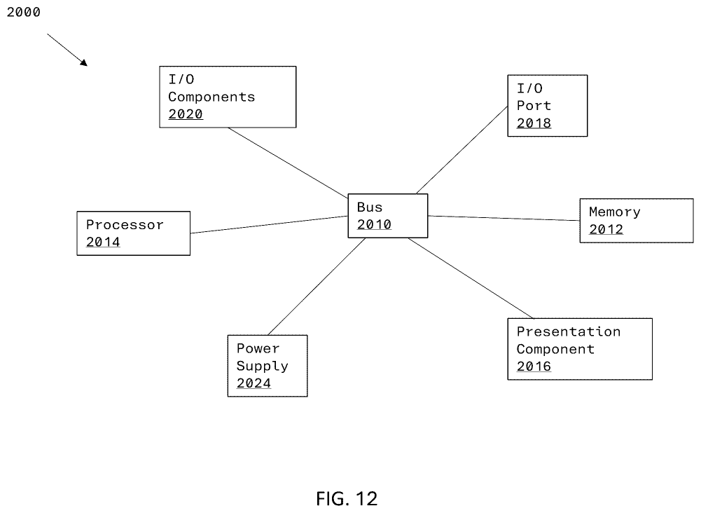

Any suitable computing systems can be used to implement the computing devices and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 2000 is depicted in FIG. 12. The computing device 2000 is merely an illustrative example of a suitable computing environment and in no way limits the scope of the present disclosure. A “computing device,” as represented by FIG. 12, can include a “workstation,” a “server,” a “laptop,” a “desktop,” a “hand-held device,” a “mobile device,” a “tablet computer,” or other computing devices, as would be understood by those of skill in the art. Given that the computing device 2000 is depicted for illustrative purposes, embodiments of the present disclosure may utilize any number of computing devices 2000 in any number of different ways to implement a single embodiment of the present disclosure. Accordingly, embodiments of the present disclosure are not limited to a single computing device 2000, as would be appreciated by one with skill in the art, nor are they limited to a single type of implementation or configuration of the example computing device 2000.

The computing device 2000 can include a bus 2010 that can be coupled to one or more of the following illustrative components, directly or indirectly: a memory 2012, one or more processors 2014, one or more presentation components 2016, input/output ports 2018, input/output components 2020, and a power supply 2024. One of skill in the art will appreciate that the bus 2010 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 12 is merely illustrative of an exemplary computing device that can be used to implement one or more embodiments of the present disclosure, and in no way limits the disclosure.

The computing device 2000 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 2000.

The memory 2012 can include computer-storage media in the form of volatile and/or nonvolatile memory. The memory 2012 may be removable, non-removable, or any combination thereof. Exemplary hardware devices are devices such as hard drives, solid-state memory, optical-disc drives, and the like. The computing device 2000 can include one or more processors that read data from components such as the memory 2012, the various I/O components 2020, etc. Presentation component(s) 2016 present data indications to a user or other device. Exemplary presentation components include a display device, speaker, printing component, vibrating component, etc.

The I/O ports 2018 can enable the computing device 2000 to be logically coupled to other devices, such as I/O components 2020. Some of the I/O components 2020 can be built into the computing device 2000. Examples of such I/O components 2020 include a microphone, joystick, recording device, game pad, satellite dish, scanner, printer, wireless device, networking device, and the like.

Embodiments of the present disclosure are set out in the below clauses.

Clause 1. A system for tracking a rigid anatomical structure in computer-assisted surgery, comprising: an acquisition device configured to acquire imaging data of a trackable surface, the trackable surface being a portion of the rigid anatomical structure, the trackable surface comprising one or more characteristic features identifiable in the imaging data; a tracking device configured to track a pose of the acquisition device with respect to a reference coordinate system; and one or more processors communicatively coupled to the acquisition device and the tracking device, configured to: receive imaging data of the trackable surface from the acquisition device; receive tracking data from the tracking device and determine a pose of the acquisition device with respect to the reference coordinate system based on the tracking data; track a pose of the one or more characteristic features of the trackable surface within the imaging data; based on the pose of the one or more characteristic features, determine a pose of the trackable surface with respect to the acquisition device; and determine a pose of the rigid anatomical structure with respect to the reference coordinate system based on the pose of the acquisition device with respect to the reference coordinate system and the pose of the trackable surface with respect to the acquisition device.

Clause 2. The system of clause 1, wherein the one or more processors is configured to determine the pose of the trackable surface with respect to the acquisition device by: determining a pose of the one or more characteristic features of the trackable surface in a first image of the trackable surface in the imaging data; determine a pose of the one or more characteristic features of the trackable surface in a second image of the trackable surface in the imaging data; and determining a transformation matrix between the pose of the one or more characteristic features in the first image and the pose of the one or more characteristic of features in the second image.

Clause 3. The system of clause 1 or clause 2, wherein the one or more characteristic features comprises an artificial feature resulting from an alteration of a surface condition of the trackable surface by at least one of the following methods: drilling, milling, burring, stamping, engraving, embossing, cauterizing, applying ink, directing structured light.

Clause 4. The system of any preceding clause, wherein the one or more characteristic features comprises a natural feature of the trackable surface, the natural feature comprising at least one of a micro-relief, a grain, a striation, a fibrosity, or a porosity.

Clause 5. The system of any preceding clause, wherein the one or more characteristic features are identifiable as image texture in the imaging data.

Clause 6. The system of any preceding clause, wherein the image texture comprises a two-dimensional graphical pattern identifiable in the imaging data, the two-dimensional graphical pattern comprising at least one of an intensity, a contrast, a gradient, or a color.

Clause 7. The system of any preceding clause, wherein the image texture comprises a three-dimensional feature identifiable in the imaging data.

Clause 8. The system of any preceding clause, wherein the acquisition device comprises at least one of: a miniaturized camera, a stereo camera, an RGB-D camera, an endoscopic camera, a LiDAR, or an ultrasonic probe.

Clause 9. The system of any preceding clause, further comprising a light-emitting device configured to illuminate the trackable surface during acquisition of the imaging data with structured light rays, the structured light rays producing a visual pattern or a polarized light.

Clause 10. The system of any preceding clause, further comprising a marker rigidly attached to the acquisition device, and wherein the one or more processors is configured to determine a pose of the marker with respect to the reference coordinate system, the pose of the acquisition device with respect to the reference coordinate system being determined based on the pose of the marker with respect to the reference coordinate system and a known pose of the marker with respect to the acquisition device.

Clause 11. The system of any preceding clause, wherein the marker comprises a first electromagnetic transducer and the tracking device comprises a second electromagnetic transducer, one of the first electromagnetic transducer and the second electromagnetic transducer being an emitter configured to generate a magnetic field, and the other electromagnetic transducer being a receiver configured to measure the magnetic field generated by the emitter, the one or more processors being configured to: receive the magnetic field measured by the receiver; and determine the pose of the marker with respect to the reference coordinate system based on the measured magnetic field.

Clause 12. The system of any preceding clause, wherein the marker comprises an optical bracket or a visual marker, and the tracking device comprises a camera configured to acquire images of the optical bracket or the visual marker, the one or more processors being configured to: receive the images of the optical bracket or the visual marker acquired by the camera; and determine the pose of the marker with respect to the reference coordinate system based on the images of the optical bracket or the visual marker.

Clause 13. The system of any preceding clause, further comprising a robotic arm, and wherein the acquisition device is rigidly attached to the robotic arm.

Clause 14. The system of any preceding clause, wherein the one or more processors is further configured to: detect a motion of the rigid anatomical structure based on a first pose of the rigid anatomical structure with respect to the reference coordinate system, and a second pose of the rigid anatomical structure with respect to the reference coordinate system, the first pose being determined prior to the second pose; and send a command to the robotic arm when a motion of the rigid anatomical structure is detected in order to move the acquisition device so that the trackable surface remains within an acquisition range of the acquisition device.

Clause 15. The system of any preceding clause, wherein the trackable surface is a portion of the rigid anatomical structure that is not expected to be modified during a proposed surgical intervention on the rigid anatomical structure.

Clause 16. The system of any preceding clause, wherein the trackable surface is spaced a distance away from a working region of the rigid anatomical structure during a proposed surgical intervention on the rigid anatomical structure.

Clause 17. The system of any preceding clause, wherein the trackable surface is sized to include a number of characteristic features sufficient to allow tracking of the trackable surface.

Clause 18. The system of any preceding clause, wherein the one or more characteristic features comprises a plurality of characteristic features.

Clause 19. The system of any preceding clause, wherein the one or more processors is further configured to: receive initial imaging data of a surface of a portion of the rigid anatomical structure; identify one or more characteristic features of the surface in the initial imaging data; determine a trackability of the one or more characteristic features of the surface; and tag the surface as the trackable surface if the one or more characteristic features of the surface are trackable.

Clause 20. A system, comprising: one or more processors; and one or more storage mediums having encoded thereon executable instructions that, when executed by the one or more processors, cause the one or more processors to: receive imaging data of a trackable surface, wherein the trackable surface: is a portion of a rigid anatomical structure to be operated on; and comprises one or more characteristic features; track a pose of the one or more characteristic features in the imaging data; and determine, based on the pose of the one or more characteristic features, a pose of the rigid anatomical structure.

Clause 21. The system of clause 20, wherein: the one or more characteristic features comprises an artificial feature resulting from an alteration of a surface condition of the trackable surface by at least one of the following methods: drilling, milling, burring, stamping, engraving, embossing, cauterizing, applying ink, or directing structured light; or the one or more characteristic features comprises a natural feature of the trackable surface, the natural feature comprising at least one of a micro-relief, a grain, a striation, a fibrosity or a porosity.

Clause 22. The system of clause 20 or clause 21, wherein the one or more characteristic features are identifiable as image texture in the imaging data, wherein: the image texture comprises a two-dimensional graphical pattern identifiable in the imaging data, the two-dimensional graphical pattern comprising at least one of an intensity, a contrast, a gradient, or a color; or the image texture comprises a three-dimensional feature identifiable in the imaging data.

Clause 23. A method for tracking a rigid anatomical structure, the method comprising: identifying a trackable surface, the trackable surface being a portion of the rigid anatomical structure, the trackable surface comprising one or more characteristic features; receiving imaging data of the trackable surface, the one or more characteristic features being identifiable in the imaging data; tracking a pose of the one or more characteristic features of the trackable surface within the imaging data; and based on the pose of the one or more characteristic features, determining a pose of the rigid anatomical structure.

Clause 24. The method of clause 23, further comprising: obtaining a trackability map, the trackability map comprising trackability scores associated with portions of the rigid anatomical structure, the trackability scores being computed based on at least one of a pre-operative planning or a three-dimensional model of the rigid anatomical structure; and identifying a location of the trackable surface on the rigid anatomical structure based on the trackability map.

Clause 25. The method of clause 23 or clause 24, further comprising: making an incision in a soft body part opposite to the rigid anatomical structure such that the trackable surface is exposed; and positioning an image acquisition device, the image acquisition device configured to acquire the imaging data, inside the incision facing the trackable surface, such that the trackable surface is in a field of view of the image acquisition device.

Clause 26. The method of any preceding clause, wherein the one or more characteristic features are identifiable as image texture in the imaging data, wherein: the image texture comprises a two-dimensional graphical pattern identifiable in the imaging data, the two-dimensional graphical pattern comprising at least one of an intensity, a contrast, a gradient, or a color; or the image texture comprises a three-dimensional feature identifiable in the imaging data.

Clause 27. The method of any preceding clause, comprising: receiving first imaging data of trackable surface; computing a detection score based on a plurality of features of the trackable surface detected within the first imaging data; and if the detection score is lower than a predetermined threshold, altering a surface condition of the trackable surface to form or enhance the one or more characteristic features of the trackable surface.

Clause 28. The method of any preceding clause, wherein the one or more characteristic features is formed or enhanced by at least one of the following methods: drilling, milling, burring, engraving, embossing, cauterizing, stamping, directing structured light, or applying ink.

It should be appreciated that the methods discussed above need not be completed in the order of steps discussed above. Multiple steps of the methods can be performed substantially simultaneously or in a different order than presented above. Similarly, multiple steps of the methods can be omitted, or additional steps could be added.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one nonlimiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y should be interpreted as meaning either “X” or “Y” individually, or both “X and Y” together.

All patents and publications mentioned in this specification are herein incorporated by reference to the same extent as if each independent patent and publication was specifically and individually indicated to be incorporated by reference.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of any appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by any appended claims and the applicable rules of law.

What is claimed is:

1. A system for tracking a rigid anatomical structure in computer-assisted surgery, comprising:
an acquisition device configured to acquire imaging data of a trackable surface, the trackable surface being a portion of the rigid anatomical structure, the trackable surface comprising one or more characteristic features identifiable in the imaging data;
a tracking device configured to track a pose of the acquisition device with respect to a reference coordinate system; and
one or more processors communicatively coupled to the acquisition device and the tracking device, configured to:
receive imaging data of the trackable surface from the acquisition device;
receive tracking data from the tracking device and determine a pose of the acquisition device with respect to the reference coordinate system based on the tracking data;
track a pose of the one or more characteristic features of the trackable surface within the imaging data;
based on the pose of the one or more characteristic features, determine a pose of the trackable surface with respect to the acquisition device; and
determine a pose of the rigid anatomical structure with respect to the reference coordinate system based on the pose of the acquisition device with respect to the reference coordinate system and the pose of the trackable surface with respect to the acquisition device.

2. The system of claim 1, wherein the one or more processors is configured to determine the pose of the trackable surface with respect to the acquisition device by:
determining a pose of the one or more characteristic features of the trackable surface in a first image of the trackable surface in the imaging data;
determine a pose of the one or more characteristic features of the trackable surface in a second image of the trackable surface in the imaging data; and
determining a transformation matrix between the pose of the one or more characteristic features in the first image and the pose of the one or more characteristic of features in the second image.

3. The system of claim 1, wherein the one or more characteristic features comprises an artificial feature resulting from an alteration of a surface condition of the trackable surface.

4. The system of claim 1, wherein the one or more characteristic features comprises a natural feature of the trackable surface, the natural feature comprising at least one of a micro-relief, a grain, a striation, a fibrosity, or a porosity.

5. The system of claim 1, wherein the one or more characteristic features are identifiable as image texture in the imaging data.

6. The system of claim 5, wherein the image texture comprises a two-dimensional graphical pattern identifiable in the imaging data, the two-dimensional graphical pattern comprising at least one of an intensity, a contrast, a gradient, or a color.

7. The system of claim 5, wherein the image texture comprises a three-dimensional feature identifiable in the imaging data.

8. The system of claim 1, wherein the acquisition device comprises at least one of: a miniaturized camera, a stereo camera, an RGB-D camera, an endoscopic camera, a LiDAR, or an ultrasonic probe.

9. The system of claim 1, further comprising a light-emitting device configured to illuminate the trackable surface during acquisition of the imaging data with structured light rays, the structured light rays producing a visual pattern or a polarized light.

10. The system of claim 1, further comprising a marker rigidly attached to the acquisition device, and wherein the one or more processors is configured to determine a pose of the marker with respect to the reference coordinate system, the pose of the acquisition device with respect to the reference coordinate system being determined based on the pose of the marker with respect to the reference coordinate system and a known pose of the marker with respect to the acquisition device.

11. The system of claim 10, wherein the marker comprises a first electromagnetic transducer and the tracking device comprises a second electromagnetic transducer, one of the first electromagnetic transducer and the second electromagnetic transducer being an emitter configured to generate a magnetic field, and the other one of the first electromagnetic transducer and the second electromagnetic transducer being a receiver configured to measure the magnetic field generated by the emitter, the one or more processors being configured to:
receive the magnetic field measured by the receiver; and
determine the pose of the marker with respect to the reference coordinate system based on the measured magnetic field.

12. The system of claim 10, wherein the marker comprises an optical bracket or a visual marker, and the tracking device comprises a camera configured to acquire images of the optical bracket or the visual marker, the one or more processors being configured to:
receive the images of the optical bracket or the visual marker acquired by the camera; and
determine the pose of the marker with respect to the reference coordinate system based on the images of the optical bracket or the visual marker.

13. The system of claim 1, further comprising a robotic arm, and wherein the acquisition device is rigidly attached to the robotic arm.

14. The system of claim 13, wherein the one or more processors is further configured to:
detect a motion of the rigid anatomical structure based on a first pose of the rigid anatomical structure with respect to the reference coordinate system, and a second pose of the rigid anatomical structure with respect to the reference coordinate system, the first pose being determined prior to the second pose; and
send a command to the robotic arm when a motion of the rigid anatomical structure is detected in order to move the acquisition device so that the trackable surface remains within an acquisition range of the acquisition device.

15. The system of claim 1, wherein the trackable surface is a portion of the rigid anatomical structure that is not expected to be modified during a proposed surgical intervention on the rigid anatomical structure.

16. The system of claim 1, wherein the trackable surface is spaced a distance away from a working region of the rigid anatomical structure during a proposed surgical intervention on the rigid anatomical structure.

17. The system of claim 1, wherein the trackable surface is sized to include a number of characteristic features sufficient to allow tracking of the trackable surface.

18. The system of claim 1, wherein the one or more characteristic features comprises a plurality of characteristic features.

19. The system of claim 1, wherein the one or more processors is further configured to:
receive initial imaging data of a surface of a portion of the rigid anatomical structure;
identify one or more characteristic features of the surface in the initial imaging data;
determine a trackability of the one or more characteristic features of the surface; and
tag the surface as the trackable surface if the one or more characteristic features of the surface are trackable.

20. A system, comprising:
one or more processors; and
one or more storage mediums having encoded thereon executable instructions that, when executed by the one or more processors, cause the one or more processors to:
receive imaging data of a trackable surface, wherein the trackable surface:
is a portion of a rigid anatomical structure to be operated on; and
comprises one or more characteristic features;
track a pose of the one or more characteristic features in the imaging data; and
determine, based on the pose of the one or more characteristic features, a pose of the rigid anatomical structure.

21. The system of claim 20, wherein:
the one or more characteristic features comprises an artificial feature resulting from an alteration of a surface condition of the trackable surface; or
the one or more characteristic features comprises a natural feature of the trackable surface, the natural feature comprising at least one of a micro-relief, a grain, a striation, a fibrosity or a porosity.

22. The system of claim 20, wherein the one or more characteristic features are identifiable as image texture in the imaging data, wherein:
the image texture comprises a two-dimensional graphical pattern identifiable in the imaging data, the two-dimensional graphical pattern comprising at least one of an intensity, a contrast, a gradient, or a color; or
the image texture comprises a three-dimensional feature identifiable in the imaging data.

23. A method for tracking a rigid anatomical structure, the method comprising:
identifying a trackable surface, the trackable surface being a portion of the rigid anatomical structure, the trackable surface comprising one or more characteristic features;
receiving imaging data of the trackable surface, the one or more characteristic features being identifiable in the imaging data;
tracking a pose of the one or more characteristic features of the trackable surface within the imaging data; and
based on the pose of the one or more characteristic features, determining a pose of the rigid anatomical structure.

24. The method of claim 23, further comprising:
obtaining a trackability map, the trackability map comprising trackability scores associated with portions of the rigid anatomical structure, the trackability scores being computed based on at least one of a pre-operative planning or a three-dimensional model of the rigid anatomical structure; and
identifying a location of the trackable surface on the rigid anatomical structure based on the trackability map.

25. The method of claim 23, further comprising:
making an incision in a soft body part opposite to the rigid anatomical structure such that the trackable surface is exposed; and
positioning an image acquisition device, the image acquisition device configured to acquire the imaging data, inside the incision facing the trackable surface, such that the trackable surface is in a field of view of the image acquisition device.

26. The method of claim 23, wherein the one or more characteristic features are identifiable as image texture in the imaging data, wherein:
the image texture comprises a two-dimensional graphical pattern identifiable in the imaging data, the two-dimensional graphical pattern comprising at least one of an intensity, a contrast, a gradient, or a color; or
the image texture comprises a three-dimensional feature identifiable in the imaging data.

27. The method of claim 23, comprising:
receiving first imaging data of trackable surface;
computing a detection score based on a plurality of features of the trackable surface detected within the first imaging data; and
if the detection score is lower than a predetermined threshold, altering a surface condition of the trackable surface to form or enhance the one or more characteristic features of the trackable surface.
